SEQUENCE LISTING (10 sequences)

SEQ ID NO: 1 - DNA, 4572 bp, Homo sapiens
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctt

SEQ ID NO: 2 - protein, 22 aa, Artificial Sequence
  Synthetic biotin target sequence tag
CGSGLNDIFEAQKIEWHEGAPC

SEQ ID NO: 3 - protein, 8 aa, Artificial Sequence
  Synthetic FLAG epitope
DYKDDDDK

SEQ ID NO: 4 - protein, 9 aa, Artificial Sequence
  Synthetic hemagglutinin tag
YPYDVPDYA

SEQ ID NO: 5 - DNA, 33 bp, Artificial Sequence
  Synthetic oligonucleotide primer
acacactcagttaaccaaggtcagaacattcac

SEQ ID NO: 6 - DNA, 57 bp, Artificial Sequence
  Synthetic oligonucleotide primer
gattttctgagcctcgaagatgtcgttcaggccggagccgcagttatttctactatg

SEQ ID NO: 7 - DNA, 60 bp, Artificial Sequence
  Synthetic oligonucleotide primer
gaggctcagaaaatcgaatggcacgaaggcgcgccgtgcagctatgcagtgattatcacc

SEQ ID NO: 8 - DNA, 48 bp, Artificial Sequence
  Synthetic oligonucleotide primer
ccagatgtcatctttcttcacgtggtaattctcaataataatcataac

SEQ ID NO: 9 - DNA, 42 bp, Artificial Sequence
  Synthetic oligonucleotide primer
ggagacaatggatccaaggataacaccgtgccactgaaattg

SEQ ID NO: 10 - DNA, 56 bp, Artificial Sequence
  Synthetic oligonucleotide primer
gatgccccaagcttggatcctcatttttctgcactacgcagggatatttcaccgcc

(19) United States
(12) Patent Application Publication
(10) Pub. No.: US 2005/0191710 A1
(43) Pub. Date: Sep. 1, 2005

(54) Method for labeling a membrane-localized protein

(76) Inventors: John W. Hanrahan, Montreal West (CA); Yishan Luo, Montreal (CA)

Correspondence Address: Licata & Tyrrell P.C., 66 E. Main Street, Marlton, NJ, 08053, US

(21) Appl. No.: 10/790,273

(22) Filed: Mar. 1, 2004

(51) Int. Cl. C12Q 1/68; G01N 33/53; C07K 14/705; C12P 21/06

(52) U.S. Cl. 435/7.5; 530/350; 435/68.1

(57) ABSTRACT

The present invention relates to a method for labeling a membrane-localized protein by introducing a biotin target sequence tag into at least one loop domain of a membrane-localized protein. The method of the invention is useful for labeling an ion channel protein such as CFTR or mutants thereof. A method for identifying an agent which corrects protein misfolding of a membrane-localized protein is also provided.

### BACKGROUND OF THE INVENTION

The ABC transporters constitute a family of membrane proteins which are highly conserved in evolution. They are involved in the translocation of various substrates through cell membranes. In mammals, many ABC transporters are associated with pathologies. For example, the cystic fibrosis transmembrane conductance regulator (CFTR) is defective in cystic fibrosis; glycoprotein P (MDR: multi-drug resistance) mediates resistance to antitumor drugs; and protein ABC1 plays an essential role in endocytosis of apoptotic bodies by the macrophage.

CFTR controls the transport of chloride ions and hydration of mucous by epithelial tissues. These are reduced by mutations in the CFTR gene. The most common cystic fibrosis mutation is the deletion of a phenylalanine residue at position 508. It is found on ˜70% of cystic fibrosis chromosomes world-wide, and >90% of patients have at least one ΔF508 allele. ΔF508 occurs within the first of two nucleotide binding folds (NBF-1; Schoumacher, et al. (1990) Proc. Natl. Acad. Sci. USA 87:4012-4016; Riordan, et al. (1979) Science 245:1066-1073).

There are many mutations in the CFTR gene besides ΔF508 that reduce cellular apical chloride (Cl⁻) conductance and cause an abnormal electrical potential difference across cystic fibrosis epithelia. Like ΔF508, many of these mutations lead to misprocessing of the nascent protein. Most of the mutant protein (>99%) is retained in the endoplasmic reticulum and degraded without ever reaching the plasma membrane. Wild-type CFTR protein is also prone to retention, with only 25-30% exiting the plasma membrane.

Recent studies indicate that CFTR interacts functionally and physically with other proteins at the cell surface. Its channel activity is inhibited when complexed with syntaxin 1A and SNAP 23 (soluble N-ethylmaleimide-sensitive factor attachment protein of 23 kD; Naren, et al. (1997) Nature 390:302-305; Cormet-Boyaka, et al. (2002) Proc. Natl. Acad. Sci. USA 99:12477-12482), and may be stimulated when its C-terminus binds EBP-50 (ezrin binding protein of 50 kD; also known as NHERF or sodium hydrogen exchanger regulatory factor; Hall, et al. (1998) Proc. Natl. Acad. Sci. USA 95, 8496-8501; Short, et al. (1998) J. Biol. Chem. 273:19797-19801; Wang, et al. (1998) FEBS Lett. 427:103-108), E3KARP (sodium hydrogen exchanger 3 kinase A regulatory protein; Sun, et al. (2000) J. Biol. Chem. 275:29539-29546) or CAP-70 (CFTR associated protein of 70 kD; Wang, et al. (2000) Cell 103:169-179). CFTR is also associated with various regulatory enzymes including a phosphatase, catalytic and Type II regulatory subunits of protein kinase A (PKA), protein kinase C-ε, and the metabolic sensor AMPKinase (Hanrahan, et al. (2003) in: ABC Proteins: From Bacteria to Man, eds. Holland, et al., Elsevier Sci. Ltd., New York, pp. 589-618). The association of CFTR within macromolecular complexes can also be regulated (Naren, et al. (2003) Proc. Natl. Acad. Sci. USA 100:342-346), nevertheless the precise nature and physiological significance of CFTR complexes remain poorly understood.

Like other membrane proteins (e.g., glutamate receptors at neuronal post-synaptic densities; Borgdorff & Choquet (2002) Nature 417:649-653), the interaction of CFTR with other proteins is expected to reduce its lateral mobility through formation of large, slowly diffusing complexes or by tethering to scaffold proteins. Indeed, the mobility of a GFP-CFTR fusion protein has been measured using fluorescence recovery after photobleaching (FRAP; Haggie, et al. (2002) J. Biol. Chem. 277:16419-16425). These fusion proteins diffused relatively rapidly (diffusion coefficient D=10⁻⁹ cm²sec⁻¹ in the endoplasmic reticulum, 10⁻¹⁰ cm²sec⁻¹ in the plasma membrane; Haggie, et al. (2004) J. Biol. Chem. 279(7):5494-500) and nearly all molecules were mobile.

There is a need in the art for a method of specifically labeling a membrane-localized protein that does not adversely affect protein-protein interactions, provides a means for affinity purification and allows for one to conduct mobility studies.

Specific biotinylation of protein termini is used for purifying soluble recombinant proteins (Cull & Schatz (2000) Methods Enzymol. 326:430-440) and a biotin acceptor domain has been attached to the C-terminus of human P-glycoprotein, to purify that protein from yeast cell lysates (Julien, et al. (2000) Biochemistry 39:75-85).

U.S. Pat. Nos. 5,723,584 and 5,874,239 disclose biotinylation peptides and methods for biotinylating a protein by coupling said protein to either the carboxyl or amino terminus of said biotinylation peptide.

### SUMMARY OF THE INVENTION

The present invention relates to a method for labeling a membrane-localized protein. It involves introducing a biotin target sequence tag into at least one loop domain of a membrane-localized protein and exposing said protein to a biotin ligase in the presence of biotin so that the membrane-localized protein becomes labeled. In one embodiment, the membrane-localized protein is an ion channel as exemplified by cystic fibrosis transmembrane conductance regulator (CFTR) and misfolded mutants thereof, which are defective in their membrane localization. An isolated recombinant CFTR protein encoded by a nucleic acid sequence of SEQ ID NO:1 containing a biotin target sequence introduced into an extracellular loop is also provided. In one embodiment, the biotin target sequence tag is introduced into loop four of CFTR protein encoded by a nucleic acid sequence of SEQ ID NO:1. The labeling method of the present invention will be useful in isolating a membrane-localized protein, in monitoring the mobility of a membrane-localized protein, and in identifying proteins which interact with the tagged membrane-localized protein.

The invention further relates to a method for identifying an agent that corrects misfolding and trafficking of a membrane-localized protein. This method of the invention involves cells that express a misfolded membrane-localized protein, wherein said protein is tagged with a biotin target sequence; contacting the cell with a test agent and a biotin ligase in the presence of biotin so that the biotin target sequence of the protein becomes enzymatically labeled with biotin; and detecting the presence of labeled protein in cells contacted with the test agent, wherein the presence of labeled protein indicates that the agent corrects protein misfolding of a membrane-localized protein. In particular embodiments of the present invention, this method further includes the step of contacting the cell with a permeabilizing agent before the step of detecting the labeled protein.

### DETAILED DESCRIPTION OF THE INVENTION

The present invention relates to specific in vivo labeling of a membrane-localized protein. It has now been shown that insertion and enzymatic biotinylation of a biotin target sequence (also referred to herein as a biotinylation target sequence) engineered into a loop domain of a membrane-localized protein enables affinity purification and mobility studies using image correlation spectroscopy (ICS). Specifically, CFTR was labeled on the cell surface and shown to have a very slow lateral diffusion (2×10⁻¹¹ cm²sec⁻¹) indicative of CFTR-protein interactions which have not been demonstrated using other CFTR labeling methods. Further, inserting the biotin target sequence had little effect on protein expression according to western blots, or channel activity as assessed by iodide efflux and patch clamp assays. Thus, it is contemplated that the present invention is widely applicable to ion channels and other proteins expressed at the cell surface or other cellular membranes.

To illustrate, a biotinylation target sequence was introduced into an extracellular loop of CFTR. The biotinylation target sequence was based on peptide #85 taught by Schatz et al. ((1993) Biotechnology 11:1138-1143), with the addition of three or four amino acid residues at the N- and C-terminal ends of the peptide. It was contemplated that the addition of a cysteine residue at the N- and C-terminus of the peptide could stabilize the resulting tagged protein by formation of a disulfide bond, i.e., forming a hairpin loop, during transit and processing through the endoplasmic reticulum.

The biotinylation target sequence used herein (Cys-Gly-Ser-Gly-Leu-Asn-Asp-Ile-Phe-Glu-Ala-Gln-Lys-Ile-Glu-Trp-His-Glu-Gly-Ala-Pro-Cys; SEQ ID NO:2) was inserted into the fourth extracellular loop of CFTR between amino acid residue Asn⁹⁰¹ and Ser902 and was about three times longer than the FLAG® epitope (Asp-Tyr-Lys-Asp-Asp-Asp-Asp-Lys; SEQ ID NO:3) (Howard, et al. (1995) Am. J. Physiol. Cell Physiol. 269:C1565-C1576) and shorter than the triple HA tag (influenza hemagglutinin, Tyr-Pro-Tyr-Asp-Val-Pro-Asp-Tyr-Ala; SEQ ID NO:4) (Benharouga, et al. (2003) J. Biol. Chem. 278:22079-22089) used in similar studies.

It is shown herein that the use of a biotinylation target sequence does not significantly affect wild-type activities of CFTR. For example, introducing the biotinylation target sequence (22 amino acids, mass ˜2.4 kD, net charge −3.16) into extracellular loop four or adding glycine and ten histidines to the C-terminus (CFTR-10His; Zhu, et al. (1999) J. Biol. Chem. 274:29102-29107) had little effect on protein expression and did not alter the relative amounts of mature vs immature protein as determined by core and complex glycosylated bands on SDS-PAGE, respectively, and sensitivity to glycosidases endoglycosidase H and amino-peptidyl-glycosidase. Further, only the core-glycosylated (immature) form of ΔF508CFTR-biotintag-10His was expressed, consistent with its misfolding and retention in the endoplasmic reticulum of cells from cystic fibrosis patients (Cheng, et al. (1990) Cell 63:827-834; Kartner, et al. (1992) Nature Genet. 1:321-326). Thus, similar to previous studies (Hammerle, et al. (2001) J. Biol. Chem. 276:14848-14854), the results provided herein demonstrate that mutations in extracellular loops generally affect the activity rather than processing of CFTR, therefore a tag inserted into the extracellular loop will be useful for detecting protein that reaches the plasma membrane.

Since CFTR functions as a chloride channel, responsiveness of the tagged channels to PKA was examined by comparing cAMP-stimulated iodide efflux from cells stably transfected with 1) wild-type CFTR, 2) CFTR with a polyhistidine tag, 3) wild-type CFTR bearing both polyhistidine and biotinylation tags or 4) a ΔF508 CFTR construct that was identical to 3) except for deletion of phenylalanine 508. Peak iodide efflux was not diminished by addind 10 histidines to the C-terminus or by insertion of the extracellular biotinylation sequence, although the latter caused a consistent delay of ˜1 minute compared to wild-type CFTR. cAMP-stimulated iodide efflux was not detected when cells expressed ΔF508CFTR-biotintag, consistent with the cystic fibrosis phenotype. Therefore, similar to epitope-tagging of the fourth extracellular loop (Benharouga, et al. (2003) J. Biol. Chem. 278:22079-22089; Schultz, et al. (1997) Am. J. Physiol. Cell Physiol. 273:C2080-C2089), iodide efflux responses to PKA were preserved after insertion of a biotinylation target sequence.

Excised CFTR-biotintag channels had approximately linear single-channel current-voltage relationships when membrane patches were bathed with symmetrical solutions, as reported previously for wild-type CFTR (Kartner, et al. (1991) Cell 64:681-691). Similar results were obtained when cells were preincubated with the biotin ligase BirA, 10 mM ATP and 400 μM biotin, although there was a small decrease in the conductance of CFTR-biotintag channels with BirA present (7.2±0.20 vs 8.0±0.17 pS; mean±s.e., p=0.013). High CFTR expression in baby hamster kidney (BHK) cells precluded analysis of single channel kinetics; however, channel gating appeared similar, indicating any effects of the biotintag on gating are subtle.

CFTR could also be purified after enzymatic biotinylation at the cell surface. BHK cells were exposed to [¹⁴C]-biotin during incubation with BirA for 10 minutes (i.e., before extensive internalization of biotinylated CFTR). The reaction was carried out at 30° C. with saturating biotin (50 μM, equilibrium dissociation constant 10-100 nM) and elevated ATP (10 mM; KM for ATP=3 mM as estimated from the initial rate of biotinyl-5′-AMP synthesis; Beckett, et al. (1999) Protein Science 8:921-929). [¹⁴C]-biotin incorporation rate became faster as BirA concentration was elevated, however increasing the enzyme concentration above 5 μg/mL had progressively less effect. Some [¹⁴C]-biotin may be taken up by cells via the sodium-dependent multivitamin transporter (Prasad, et al. (1998) J. Biol. Chem. 273:7501-7506) and/or constitutive fluid-phase endocytosis under these conditions in addition to its reaction with CFTR-biotintag. No fluorescent streptavidin staining was detected on cells that expressed CFTR without the biotintag, indicating little, if any, biotin adsorption on the cell surface. The dependence on BirA exposure time was inferred from the amount of CFTR captured on streptavidin beads, assuming only biotinylated protein would bind. Maximal recovery of CFTR was achieved when cells were exposed to BirA for ˜40 minutes, therefore 5 μg/mL BirA and 40-60 minutes were used to label CFTR in subsequent experiments. The time-course of biotinylation on live cells was similar to that reported previously for soluble proteins. In previous biochemical studies of the biotinylated target sequence used herein, 10 nmol target sequence was biotinylated by 2.5 μg BirA in 250 μl reaction buffer at 30° C. after 30-40 minutes (Cull & Schatz (2000) supra). For comparison, the relative amounts of bound and unbound CFTR-biotintag after chemical biotinylation with sulfo-NHS-X-X-biotin were determined. Much less CFTR-biotintag was recovered by this commonly-used method, probably due to the low rate of the uncatalyzed reaction and low temperature (0° C.).

Capture of enzymatically biotinylated CFTR was compared with metal chelate chromatography on Ni²⁺-NTA and with immuno-purification on M3A7-protein G beads. The same construct (CFTR-biotintag-10his) and cell area (33 cm²) were used for all three methods. The yield on streptavidin beads was similar to that obtained by the other methods; slightly lower than Ni²⁺-NTA, but higher than immunopurification. CFTR was not captured from cells that expressed CFTR with biotin acceptor sequence #42 (Schatz (1993) supra) inserted at the same position, which served as a negative control. The purity of CFTR isolated from crude membranes on streptavidin, protein G or Ni²⁺-NTA beads was also compared. Biotinylation and streptavidin binding were highly specific, as evidenced by the low background in silver stained SDS-PAGE gels after purification on streptavidin beads. All three methods recovered only a fraction of the CFTR, however, these results demonstrate that enzymatic biotinylation at the cell surface can be used to isolate CFTR and will be useful in identifying CFTR-associated proteins from cell lysates and crude membranes.

Cells expressing wild-type or ΔF508 CFTR were compared by immunofluorescence staining to determine whether mislocalization of the ΔF508 mutant is preserved after insertion of the biotintag. Subconfluent cultures were fixed, permeabilized, and exposed to a monoclonal antibody specific against the R domain of CFTR (450; Jensen, et al. (2000) Ped. Pulmonol. suppl. 20:179). Cells expressing wild-type CFTR-biotintag had sharp edges consistent with expression at the plasma membrane. By contrast, cell margins were diffuse and there was little peripheral staining in cells expressing ΔF508CFTR-biotintag. This indicates that wild-type and mutant CFTR channels are distributed normally and are not perturbed by insertion of the biotintag, thus ΔF508CFTR-biotintag is useful for monitoring correction of the folding defect in high-throughput drug screens. Relatively strong intracellular staining of wild-type CFTR in permeabilized cells is likely due to overexpression by BHK cells.

Confocal images of live cells expressing wild-type CFTR-biotintag were collected at 1 Hz after incubating cells with BirA and exposure to streptavidin-Alexa568 to study the lateral diffusion of CFTR. Only the edges of cells were fluorescent in these unpermeabilized cells. The discrete correlation function for intensity fluctuations calculated from a time series of >100 images was determined and the best-fit curve was obtained using a model for two dimensional diffusion using equation 3 provided herein. The τ_{d} obtained from the fitted curve (17.7 seconds) corresponds to a diffusion coefficient D=2.2×10⁻¹¹ cm² sec⁻¹. The mean diffusion coefficient was similar at room temperature (2.4±0.3×10⁻¹¹ cm²sec⁻¹) and at 37° C. (2.0±0.2×10⁻¹¹ cm²sec⁻¹ Mean±S.E., n=20 cells, p<0.05) indicating little effect over this range. Calculated diffusion coefficients were uniform whereas the immobile fraction estimated from the best-fit temporal offset parameter was highly variable, ranging from 0 to 43%. This may reflect stable binding of CFTR to scaffolding or cytoskeletal proteins. The discrete autocorrelation function decayed very slowly when cells were pre-treated with 2% paraformaldehyde, consistent with crosslinking of CFTR into larger, less mobile complexes. Because CFTR is internalized at a rate of ˜5% per minute at 37° C. (Lukacs, et al. (1997) Biochem. J. 328:353-361) and may enter endosomes, lysosomes and other compartments involved in its recycling or degradation during the biotinylation reaction, the specificity of CFTR-biotintag labeling for the cell surface is due to binding of fluorescent streptavidin at low temperature rather than the biotinylation reaction itself. Thus, CFTR complexes captured on streptavidin beads could be analyzed for interacting proteins involved in its regulation at the plasma membrane or degradation without contamination by proteins that associate with immature (i.e., unbiotinylated) CFTR.

The conventional method for biotinylating plasma membrane proteins involves reacting a N-hydroxysulfosuccinimide ester of biotin (sulfo-NHS-biotin) with primary amines. There are eight such primary amines on the extracellular surface of CFTR-biotintag and only one substrate lysine for BirA, however the biotin target sequence tag of the present invention is advantageous over the conventional method because labeling of the biotin target sequence tag of the present invention is much faster due to the catalyst and higher temperature used (30° C. vs chemical biotinylation at 0° C.).

Further, the tagging method of the present invention is advantageous over conventional N- an C-terminal tagging as introducing the biotin target sequence into a loop domain does not disrupt interactions between the termini of the tagged protein and other regulatory proteins. For example, recent FRAP studies of GFP fused to the N-terminus of CFTR indicate rapid diffusion in the endoplasmic reticulum (diffusion coefficient 10⁻⁹ cm²/sec; Haggie, et al. (2002) J. Biol. Chem. 277:16419-16425). A diffusion coefficient of 10⁻¹⁰ cm²/sec was obtained in the plasma membrane, which is slower but still about 5-fold more rapid than calculated by image correlation spectroscopy disclosed herein. Although different analytical methods were used, FRAP experiments with biotinylated CFTR indicate that this is not the reason for the different mobilities. Rather, it is possible that Madin Darby canine kidney cells (Haggie, et al. (2002) supra) vs the baby hamster kidney cells used herein may have different sets of interacting proteins that allow different CFTR mobilities. Alternatively, protein interactions with CFTR may be partially disrupted by attachment of GFP at the amino terminus; regulatory interactions between the amino terminus of CFTR and a t-SNARE complex have been identified (Naren & Kirk (2000) News Physiol. Sci. 15:57-61).

Moreover, membrane-localized proteins, wherein both termini are on the intracellular side of the membrane, can be labeled on the extracellular surface or in intracellular organelles without disruption of the membrane of the cell or organelle to introduce the labeling agent by adding recombinant biotin ligase to the bath or targeting its expression to the lumen of the secretory pathway. In other words, the cell does not need to be permeabilized to allow intracellular access of, for example, a fluorescent label used when detecting the tag, as must currently be done with N- and C-terminal tags. Thus, protein-protein interactions and mobility of membrane-localized proteins can be analyzed in intact, live cells without disrupting interactions with cytoplasmic proteins. To illustrate, imaging the fluorescence of extracellularly-bound streptavidin to biotinylated CFTR is unlikely to interfere with protein-protein interactions at the cytoplasmic domains, which in CFTR constitute ˜95% of the exposed polypeptide. Such protein-protein interactions probably determine lateral mobility as occurs with other ion channels and receptors (Meier, et al. (2001) Nature Neuroscience 4:253-260; Tardin, et al. (2003) EMBO J. 22:4656-4665). For example, associations with PDZ domain-containing proteins causes clustering and immobilization of K channels (Burke, et al. (1999) J. Gen. Physiol. 113:71-80), and such interactions with AMPA receptors may be regulated (Borgdorff & Choquet (2002) Nature 417:649-653). Thus, CFTR mobility could reflect interactions with EBP-50, CAP70 and with regulatory kinases and phosphatases that may increase (ezrin/PKAII, RACK1/PKCε) or decrease (syntaxin 1A, type 2C protein phosphatase, AMP kinase) its channel activity (Hanrahan, et al. (2003) in: ABC Proteins: From Bacteria to Man, eds. Holland, et al., Elsevier Sci. Ltd., New York, pp. 589-618; Naren & Kirk (2000) News Physiol. Sci. 15:57-61; Schwiebert, et al. (1999) Physiol. Rev. 79:S145-S166). Indeed, deleting the C-terminal PDZ binding motif of a GFP-CFTR fusion protein increased mobility, although there was no immobilized fraction and its diffusion was much faster than reported herein (Haggie, et al. (2004) supra).

One embodiment of the present invention is a method for labeling a membrane-localized protein. As used herein, a membrane-localized protein is one which has at least two membrane-spanning domains which create at least one loop domain or region on one side of the membrane (i.e., intracellular or extracellular with regard to the plasma membrane or intralumenal or extralumenal with regard to an organelle). The method involves introducing or incorporating a biotin target sequence tag into at least one loop domain of a membrane-localized protein and exposing said protein to a biotin ligase in the presence of biotin so that the membrane-localized protein is labeled.

A membrane-localized protein can be a protein which is only transiently associated with the membrane or a protein which has a low rate of turnover in the membrane. For the purposes of the present invention, a membrane-localized protein can be located or associated with the membrane of a cell or organelle (e.g. inner or outer membrane of the mitochondria or nucleus, lysosomal membrane, endoplasmic reticulum, Golgi apparatus, vacuole, peroxisome, or plastid) of a mammal such as a human, or other organism such as a fungus, bacterium, plant, or protozoan. As one of skill in the art can appreciate, the method of the invention can be used to label any membrane-localized protein so that said protein can be purified, localized, have its mobility monitored, etc. For example, the movement of a mitochondrial membrane-localized protein to and from inner/outer membrane junctions can be monitored without disrupting possible protein-protein interactions responsible for said movement.

Exemplary membrane-localized proteins which can be labeled in accordance with the method of the invention include, but are not limited to, ABC transporters proteins or ion channels such as ABC1, ABCR, CFTR, MRP2, SUR1, MDR3, ALD, rhodopsins, G protein-coupled receptors, porins, respiratory proteins, and the like. Further, as will be described in detail herein, mutants of an above-referenced protein which have defects in folding or localization can be tagged in accordance with the method of the invention and be used to identify agents which correct misfolding or mislocalization of the protein.

It is contemplated that extracellular (or organellar luminal) as well as intracellular (or organellar extralumenal) loops can be tagged using a biotin target sequence as disclosed herein. Labeling and detection of an extracellular loop domain in vivo has now been demonstrated with CFTR and it is contemplated that an intracellular or intralumenal loop can be tagged for detection on a membrane which has been turned inside out or, alternatively by permeabilizing the membrane of the cell or organelle to facilitate uptake of the label.

A biotin target sequence tag (i.e., a sequence recognized and specifically biotinylated by a biotin ligase or other enzyme which biotinylates a protein or peptide) can include the target sequence of SEQ ID NO:2 or variations thereof, a BCCP biotin acceptor peptide (Beckett, et al. (1999) Protein Sci. 8(4):921-9), transcarboxylase biotin acceptor domain (Howard and Roepe (2003) Biochemistry 42(12):3544-55) or sequences disclosed in U.S. Pat. No. 5,723,584, incorporated herein by reference in its entirety.

A biotin target sequence is introduced or incorporated into a suitable loop domain of a membrane-localized protein of interest using recombinant nucleic acid methods, e.g., PCR amplification or overlap PCR, restriction enzyme digestion and ligation, or by chemical synthesis methods well-known in the art. A suitable loop domain is an amino acid sequence in a membrane-localized protein which is not located at the N-terminus, C-terminus, or in a membrane-spanning region of said protein and, upon insertion of a biotin target sequence, does not disrupt the ability of said protein to be integrated into the membrane (i.e., the protein is processed and sorted properly). As will be appreciated by one of skill in the art, suitable loop domains can be experimentally determined, provided by the art, or can be based upon the location of known mutations which do not effect the ability of the protein to be integrated into the membrane.

A loop domain can be identified by plotting the hydrophobicity or hydrophilicity of a protein. The importance of the hydropathic amino acid index in conferring interactive biologic function on a protein is generally understood in the art (see, Kyte and Doolittle (1982) J. Mol. Biol. 157:105). It is accepted that the relative hydropathic character of the amino acid contributes to the secondary structure of the resultant protein, which in turn defines the interaction of the protein with, for example, membranes, substrates, receptors, antibodies, antigens, and the like.

Each amino acid has been assigned a hydropathic index on the basis of its hydrophobicity and charge characteristics (Kyte and Doolittle (1982) supra), and these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is also understood in the art that folding and membrane interactions of a protein can be made on the basis of hydrophilicity. U.S. Pat. No. 4,554,101 teaches that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

A tagged protein of the invention is subsequently expressed in a host cell (e.g., a mammalian, bacterial, fungal, or plant cell) using its endogenous promoter, or a regulated or constitutive promoter so that it is expressed and integrated into a membrane. Methods of introducing recombinant nucleic acid sequences into a cell are well-established in the art. For example, the coding sequence of a tagged protein of the invention can be delivered to cells using mechanical methods, such as microinjection, liposome-mediated transfection, electroporation, or calcium phosphate precipitation. Alternatively, if it is desired that the cells stably retain the construct, it can be supplied on a plasmid or vector (e.g., a viral vector) and maintained as a separate element or integrated into the genome of the cells, as is known in the art. See, e.g., Molecular Biology, Ausubel, F. M. et al. (eds.) Greene Publishing Associates, (1989), and other standard laboratory manuals. The construct can include species appropriate transcriptional regulatory elements, such as a promoter element, an enhancer or UAS element, and a transcriptional terminator signal, for controlling transcription of the coding sequence in the cells.

Exposure of a tagged protein of the invention to a biotin ligase in the presence of biotin can be carried out in vivo or in vitro, i.e., the tagged protein can be isolated, in a crude cell-membrane extract, or may be located in the membrane of an intact cell or organelle. Further, the biotin ligase can be an isolated enzyme which is applied to an isolated tagged protein, membrane fraction, cell or organelle. Alternatively, the biotin ligase can be co-expressed in the cell which contains the tagged protein. When the tagged protein is localized to the plasma membrane, the biotin ligase can contain any well-known signal sequence which directs the ligase to be secreted or which tethers the ligase to the plasma membrane. In this manner, the ligase will be transported through the secretory system with the tagged protein so that during transport the ligase labels the tagged protein with biotin which is found within the secretory system. Alternatively, the ligase can be fused to an endoplasmic reticulum retention signal such as HDEF, HDEL, KDEL, RDEL, or KEEL so that it is retained in the endoplasmic reticulum and biotinylates secreted tagged proteins. If a protein is tagged on an intracellular or extralumenal loop domain or processed in the cytoplasm, a biotin ligase can be expressed in the cytoplasm. An exemplary biotin ligase which may be used to carry out the method of the invention includes, but is not limited to, BirA, the nucleotide sequence of which is well-known in the art (e.g., accession number M15820).

A biotin can be biotin, a tritiated biotin, a biotin analog such as 2-iminobiotin or a biotin derivative such as fluorescein biotin, biotin-4-fluorescein, biotin-X, lucifer yellow biocytin, Alexa Fluor 488 biocytin, Alexa Fluor 546 biocytin, Alexa Fluor 594 biocytin, or Oregon Green 488 biocytin (Molecular Probes, Eugene, Oreg.). Detection of a biotinylated membrane-localized protein can be performed using any of the well-known avidin or streptavidin reagents or by directly detecting a tritiated or fluorescent biotin derivative. Detection of biotin-avidin or biotin-streptavidin complexes typically involves conjugated forms of avidin or streptavidin including, but are not limited to, enzyme-conjugates (e.g., alkaline phosphatase, β-galactosidase, glucose oxidase, horseradish peroxidase) or fluorescent-conjugates (e.g., 7-amino-4-methylcoumarin-3-acetic (AMCA), fluorescein, phycoerythrin, rhodamine, TEXAS RED®, OREGON GREEN®) or antibodies which specifically bind to avidin or streptavidin. Methods of detecting antibodies are well-known to those of skill in the art (see, e.g., “Methods in Immunodiagnosis”, 2nd Edition, Rose, and Bigazzi, eds. John Wiley & Sons, 1980; Campbell et al., “Methods and Immunology”, W.A. Benjamin, Inc., 1964; and Oellerich, M. (1984) J. Clin. Chem. Clin. Biochem. 22:895-904). In certain applications it is desirable that the label to be imaged is fluorescent, i.e, an avidin or streptavidin conjugated to fluorescent label.

Methods of imaging and analyzing any of the above-mentioned labels are well-known in the art and the method employed will vary with the type of analysis being conducted, i.e. individual samples or multiple sample analyses in high-throughput screens. Measurement of the label can be accomplished using methods disclosed herein as well as flow cytometry, spectrofluorometer, fluorescence microscopy, fluorescence scanners and the like.

Another embodiment of the present invention is a method for identifying an agent which corrects protein misfolding of a membrane-localized protein. It is well-known in the art that various diseases are the result of misfolding or mislocalization of a membrane-localized protein. Accordingly, an agent which corrects the folding or localization defect of such a protein would be useful in treating such a disease. As a first step in the screening method of the invention, a test cell which expresses a misfolded membrane-localized protein is obtained. In order to carry out the screen, the membrane-localized protein is tagged with a biotin target sequence so that it can be detected in subsequent steps. Methods of tagging a membrane-localized protein with a biotin target sequence and expressing said protein in a cell are disclosed herein. While it may be desirable to express the tagged, misfolded protein in a cell or cell line in which the misfolded protein is normally found, it may in certain cases be advantageous or desirable to express the tagged, misfolded protein in a cell or cell line from another species or model system. For example, it is contemplated that a tagged, misfolded human protein can be expressed in mouse or yeast cells using appropriate regulatory sequences and vectors which are well-known in the art for such cells.

Exemplary misfolded membrane=localized proteins which may be tagged and used in accordance with the screening method of the invention include, but are not limited to, ABC1 mutants which cause familial high density lipoprotein deficiency (FHD); ABCR mutants which cause Stargardt disease; MDR3 mutants which cause progressive familial intrahepatic cholestasis (PFIC) or intrahepatic cholestasis of pregnancy (ICP); MRP2 mutants which cause Dubin-Johnson Syndrome; SUR1 mutants which cause persistent hyperinsulinemic hypoglycemia of infancy (PHHI); ALD mutants which cause X-linked adrenoleukodystrophy; rhodopsin mutants which cause autosomal dominant retinitis pigmentosa; vasopressin receptor or aquaporin water channel mutants which cause congenital nephrogenic diabetes insipidis; or CFTR mutants which cause cystic fibrosis. Mutations of CFTR known to cause protein misfolding, include but are not limited to, H139R, G149R, D192G, and R258G (Seibert, et al. (1997) Biochemistry 36:11966-11974); S945L and H949Y (Seibert, et al. (1996) J. Biol. Chem. 271:27493-27499); H1054, G1061R, L1065P, R1066C, R1066H, R1066L, Q1071P, L1077P, H1085R, W1098R, M1101K, and M1101R (Seibert et al. (1996) J. Biol. Chem. 271:15139-15145), and other mutations well-known in the art (see, e.g., Cystic Fibrosis Genetic Analysis Consortium (1994) Hum. Mutat. 4(3):167-77).

The second step of the screening method of the present invention involves contacting or adding at least one test agent to a point of application, such as a well, in a plate containing the test cell and incubating the plate for a time sufficient to allow the test agent to effect protein folding or localization.

Agents which correct protein misfolding of a membrane-localized protein can be rationally designed from the crystal structure of the protein of interest or identified from a library of test agents. Test agents of a library can be synthetic or natural compounds. A library can comprise either collections of pure agents or collections of agent mixtures. Examples of pure agents include, but are not limited to, peptides, polypeptides, antibodies, oligonucleotides, carbohydrates, fatty acids, steroids, purines, pyrimidines, lipids, synthetic or semi-synthetic chemicals, and purified natural products, derivatives, structural analogs or combinations thereof. Examples of agent mixtures include, but are not limited to, extracts of prokaryotic or eukaryotic cells and tissues, as well as fermentation broths and cell or tissue culture supernatants. In the case of agent mixtures, one may not only identify those crude mixtures that possess the desired activity, but also monitor purification of the active component from the mixture for characterization and development as a therapeutic drug. In particular, the mixture so identified can be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained.

Agents of interest in the present invention are those with functional groups necessary for structural interaction with proteins, particularly hydrogen bonding, and typically include at least an amine, carbonyl, hydroxyl or carboxyl group. The agents often comprise cyclical carbon or heterocyclic structures and/or aromatic or polyaromatic structures substituted with one or more of the above functional groups.

Subsequent to, or simultaneously with the addition of the test agent, a biotin ligase is added to the test cell in the presence of biotin so that the biotin target sequence of the protein is labeled (i.e., biotinylated). In general, this step is carried out by contacting the test cell with an exogenous source of biotin ligase and biotin. Alternatively, biotin ligase can be co-expressed within the cell along with the tagged protein of interest. Accordingly, only protein which is correctly folded and localized to the plasma membrane will be labeled and detected.

The step of detecting the presence of labeled protein in cells contacted with the test agent can be carried out as described herein, e.g., using a fluorescent biotin derivative or avidin/streptavidin conjugates. An agent which corrects folding of a misfolded membrane-localized protein will facilitate the integration and therefore labeling of the protein by biotin at the membrane. In other words, the presence of labeled protein indicates that the agent corrected misfolding of the membrane-localized protein. Localization of the correctly folded, biotinylated protein on the plasma membrane can be directly detected in a living cell, however it is contemplated that when an avidin or streptavidin is used to detect proteins which have a high turnover, the levels of detectable plasma membrane-localized proteins may be low. Accordingly, to increase the sensitivity of the screening assay, the test cells exposed to the exogenous biotin ligase are contacted with a permeabilizing agent before detection of the biotinylated protein with an avidin or streptavidin, so that avidin or streptavidin is taken up by cells and binds to internalized biotinylated proteins that had previously reached the cell surface, as well as the plasma membrane-localized biotinylated proteins. Alternatively, the sensitivity of the screening assay can be increased without permeabilizing the test cell by using a fluorescent biotin which will label both internalized proteins that had previously reached the cell surface and plasma membrane-localized tagged proteins.

Permeabilization can be carried out at a temperature ranging from approximately 4° C. to 37° C. C for a period of time from approximately 10 minutes to 60 minutes. An exemplary fixing and permeabilizing agent is paraformaldehyde (e.g., at a concentration ranging from 2% to 4%), however, as one of skill in the art will appreciate, other reagents including, but not limited to, chilled methanol (100%), TRITON™ X-100 (e.g., 0.1%-l. %), digitonin (e.g., 30 μg/ml-40 μg/ml), and saponin (e.g., 0.05%-0.25%) can also be used to permeabilize cells after mild fixation with 1.0% or lower concentration of paraformaldehyde. The extent of permeabilization of a cell by a permeabilizing agent may vary and is dependent on factors such as cell type, culture medium, and temperature. A cell is said to be permeabilized if the avidin/streptavidin is taken up by the cell in an amount sufficient to bind and detect intracellular biotinylated protein. Permeabilization can also be determined using other well-known methods such as phalloidin uptake.

Screening assays of the invention can be performed in any format that allows rapid preparation and processing of multiple reactions such as in, for example, multi-well plates of the 96-well variety. Stock solutions of the agents as well as assay components are prepared manually and all subsequent pipeting, diluting, mixing, washing, incubating, sample readout and data collecting is done using commercially available robotic pipeting equipment, automated work stations, and analytical instruments for detecting the signal generated by the assay.

In addition to the test agent and test cell, a variety of other reagents can be included in the screening assays. These include reagents like phosphate donors such as ATP, salts, neutral proteins, e.g., albumin, detergents, etc. Also, reagents that otherwise improve the efficiency of the assay, such as protease inhibitors, nuclease inhibitors, anti-microbial agents, and the like can be used.

Agents identified in accordance with the screening assays provided herein are useful in correcting folding defects of a membrane-localized protein and are therefore useful in treating diseases associated with the misfolding of said membrane-localized protein. Accordingly, agents identified herein can be formulated into pharmaceutical compositions comprising an effective amount of the active compound and a pharmaceutically acceptable vehicle. Such pharmaceutical compositions can be prepared by methods and contain vehicles which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. For example, sterile saline and phosphate-buffered saline at physiological pH may be used. Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used. Liposomes, such as those described in U.S. Pat. No. 5,422,120, WO 95/13796, WO 91/14445, or EP 524,968 B1, may also be used as a carrier.

By effective amount it is meant an amount of active compound which corrects the folding defect of a membrane-localized protein and eliminates, reduces or alleviates at least one sign or symptom of a disease associated with the misfolding of said membrane-localized protein.

A pharmaceutical composition of the invention can be administered to a cell or subject, such as a human, by any suitable means, including parenteral injection (such as intraperitoneal, subcutaneous, or intramuscular injection), orally, or by topical application (e.g., transdermal or via a mucosal surface).

The invention is described in greater detail by the following non-limiting examples.

### EXAMPLE 1

### Construction and Stable Expression of CFTR-Biotintag in Mammalian Cells

Nucleotide sequences encoding peptides that resemble those previously disclosed (i.e., peptides #42 and #85 of Schatz (1993) supra) were inserted into pNUT-CFTR using standard protocols (Sambrook, et al. (1989) Molecular Cloning: A Laboratory Manual (Cold Spring Harbor Laboratory Press, Plainview, N.Y.)). Two biotinylation target sequences were inserted at multiple locations in the polypeptide. An nsertion after amino acid 901 of CFTR protein (encoded by a nucleic acid sequence of SEQ ID NO:1), a location in the fourth extracellular loop that was shown previously to tolerate insertion of epitope tags (Howard, et al. (1995) supra; Benharouga, et al. (2003) supra), are described herein. Peptide #42 and an extended version of #85 (original peptide underlined; Cys-Gly-Ser-Gly-Leu-Asn-Asp-Ile-Phe-Glu-Ala-Gln-Lys-Ile-Glu-Trp-His-Glu-Gly-Ala-Pro-Cys; SEQ ID NO:2) were tested, however only the latter sequence was biotinylated. A DNA fragment encoding 11 N-terminal amino acids of the biotin target sequence and upstream CFTR residues was generated by the polymerase chain reaction (PCR) using wild-type CFTR cDNA as the template and forward and reverse primers CB1 (fwd: 5′-ACA CAC TCA GTT AAC CAA GGT CAG AAC ATT CAC-3′; SEQ ID NO:5) and CB2 (rev: 5′-GAT TTT CTG AGC CTC GAA GAT GTC GTT CAG GCC GGA GCC GCA GTT ATT TCT ACT ATG-3′; SEQ ID NO:6), respectively. Underlined bases indicate the HpaI endonuclease restriction site at nucleotide 2464 of the CFTR cDNA (Riordan, et al. (1989) Science 245:1066-1073). A fragment encoding the distal 13 amino acids of the biotinylation sequence and downstream CFTR residues was generated using primers CB3 (fwd: 5′-GAG GCT CAG AAA ATC GAA TGG CAC GAA GGC GCG CCG TGC AGC TAT GCA GTG ATT ATC ACC-3′; SEQ ID NO:7) and CB4 (rev: 5′-CCA GAT GTC ATC TTT CTT CAC GTG GTA ATT CTC AAT AAT AAT CAT AAC-3′; SEQ ID NO:8). Underlined bases in CB4 denote the PmlI site at position 3724. Products were joined by PCR overlap using CB1 and CB4, and sub-cloned to generate pNUT-CFTR-biotintag. The same approach was used to prepare pNUT-ΔF508CFTR-biotintag (a disease mutation) and pNUT-CFTR-biotintag-His10 (for purification on nickel-NTA beads). Constructs were transfected into baby hamster kidney (BHK) cells by calcium phosphate coprecipitation and stable lines selected using 500 μM methotrexate in accordance with well-established methods (Chang, et al. (1993) J. Biol. Chem. 268:11304-11311; Chappe, et al. (2003) J. Physiol. 548:39-52).

### EXAMPLE 2

### Cloning, Expression and Purification of BirA from E. coli

Biotinylation of CFTR on intact cells required large amounts of BirA in the extracellular medium, thus nucleic acid sequences encoding the BirA enzyme were cloned from E. coli and the BirA protein was expressed as a GST fusion to facilitate purification on SEPHAROSE 4B beads. Incubating beads with thrombin to cleave only the BirA portion yielded a band with an apparent Mr ˜30 kD, close to that expected for pure BirA. About 0.2 mg of BirA (sufficient for 20-40 reactions) was obtained from one overnight culture of recombinant cells (250 mL).

To clone the gene encoding BirA, nucleic acid sequences encoding BirA were amplified from E. coli cells by PCR using forward and reverse primers (5′-GGA GAC AAT GGA TCC AAG GAT AAC ACC GTG CCA CTG AAA TTG-3′; SEQ ID NO:9) and (5′-GAT GCC CCA AGC TTG GAT CCT CAT TTT TCT GCA CTA CGC AGG GAT ATT TCA CCG CC-3′; SEQ ID NO:10), respectively. The products were cloned into pGEX-2T (Pharmacia, Piscataway, N.J.) at the BamHI site (Naren, et al. (1997) Nature 390:302-305). After transformation into E. coli (BL21) cells and confirmation by dideoxy sequencing, 250 mL bacterial cultures were grown in LBA medium supplemented with ampicillin (Sigma, St. Louis, Mo.) to an optical density (OD, at 600 nm) of 0.6, induced for 5-6 hours at 30° C. with IPTG (0.5 mM final concentration), and harvested at OD₆₀₀=1.4. The bacterial pellet was washed twice with phosphate-buffered saline (PBS), resuspended in 25 mL PBS containing protease inhibitors and sonicated twice (Vibra Cell Sonics & Materials Inc., Danbury, Conn.). TRITON X-100 was added to a final concentration of 1% and incubated for 30 minutes at 4° C. After centrifugation at 31,000×g, the supernatant was adsorbed onto glutathione SEPHAROSE 4B beads (200 μL bed volume) that had been pre-equilibrated with PBS. Beads were washed five times with 10 volumes of cold PBS and incubated overnight at 4° C. in 1 mL PBS containing 50 units thrombin to release only BirA, which was collected by centrifuging at 1000×g for 5 minutes. Concentration and purity were assessed by SDS-PAGE.

### EXAMPLE 3

### Iodide Efflux and Patch Clamp Studies of CFTR-Biotintag

Iodide effluxes were measured using standard methods (Cormet-Boyaka, et al. (2002) Proc. Natl. Acad. Sci. USA 99:12477-12482; Cormet-Boyaka, et al. (2002) Proc. Natl. Acad. Sci. USA 99:12477-12482). Briefly, cells were incubated with iodide loading buffer (136 mM NaI, 3 mM KNO₃, 2 mM Ca(NO₃)₂, 11 mM glucose and 20 mM HEPES, pH 7.4) for 1 hour at room temperature. Extracellular NaI was removed by rinsing with iodide-free efflux buffer (same as loading buffer except NaNO₃ replaced NaI). Samples were collected by removing the efflux buffer at 1 minute intervals and replacing it with fresh solution. The first three samples established the baseline efflux rate, then cpt-cAMP was added and samples were collected at 1 minute intervals in the continuous presence of cpt-cAMP for 15 minutes. Iodide was measured using an iodide sensitive electrode (Orion Research, Inc., Boston, Mass., USA) and converted to nmoles released/minutes.

Channel activity was recorded from inside-out patches with the pipette potential held at +30 mV (i.e., membrane potential Vm=−30 mV). Pipettes were fabricated using a conventional two-stage puller (PP-83, Narishige Instrument Co., Tokyo, Japan) and had resistances of 4-6 M when filled with 150 mM NaCl solution (154 mM total [Cl]). Bath and pipette solutions initially contained (150 mM NaCl, 2 mM MgCl2 and 10 mM TES, pH 7.4). The bath was grounded through an agar bridge having the same ionic composition as the pipette. Experiments were carried out at room temperature (˜23° C.). Currents were amplified (Axopatch 1C, Axon Instruments, Foster City, Calif.), low-pass filtered using a 8-pole Bessel-type filter (902 LPF, Frequency Devices, Haverhill, Mass.) and recorded on hard disk using p-CLAMP version 8.2 software (Axon). Single channel current amplitude was taken as the difference between the mean current immediately before and during each opening, with each mean based on at least 50 samples. Several transitions were measured at each holding potential and experiments were performed on at least four patches under each set of conditions.

### EXAMPLE 4

### Immunolocalization of Wild-Type and ΔF508 CFTR-Biotintag in Fixed, Permeabilized Cells

Cells were cultured on glass coverslips to 70% confluence and fixed for 20 minutes at room temperature in 2% paraformaldehyde/PBS. After incubation for 30 minutes in PBS containing 1% bovine serum albumin (BSA) and 0.1% TRITON X-100, they were rinsed with PBS and exposed to 0.1% TRITON X-100 and monoclonal CFTR antibody (mAb 450 diluted 1:1000 in PBS; Jensen, et al. (2000) Ped. Pulmonol. suppl. 20:179) for 1 hour at room temperature. Cells were rinsed with fresh PBS/0.1% TRITON X-100 and incubated with 100 μL goat anti-mouse antibody (10 μg/mL) conjugated to Cy3 (Jackson ImmunoResearch Inc., West Grove, Pa.) in PBS/0.1% TRITON X-100 in the dark at 20° C. for 1 hour. Cells were washed three times with PBS/0.1% TRITON X-100, mounted, dried, and viewed using a Zeiss LSM 510 Confocal Laser Scanning Microscope.

### EXAMPLE 5

### Enzymatic Biotinylation at the Cell Surface

Cells were washed twice with PBS and incubated for ≦1 hour at 30° C. in 1 mL PBS supplemented with 50 mM bicine (pH 8.3), 10 mM magnesium acetate, 10 mM ATP, 400 μM biotin, and BirA (4-5 μg unless indicated otherwise). For non-specific (i.e., chemical) biotinylation, cells were washed with PBS and borate buffer (154 mM NaCl, 7.2 mM KCl, 1.8 mM CaCl₂, 10 mM boric acid, 0° C.), then incubated on ice for 15 minutes in 3 mL borate buffer containing sulfo-NHS-SS-biotin (0.5 mg/mL).

### EXAMPLE 6

### Affinity Purification of CFTR

After exposure to BirA, confluent cells in a 65-mm culture dish were rinsed three times with PBS and solubilized for 15 minutes at 4° C. in RIPA buffer (150 mM NaCl, 1 mM Tris-Cl, 1% deoxycholic acid (w/v), 1% TRITON X-100 (w/v), and 0.1% SDS) containing protease inhibitors. The lysate was centrifuged (32,000×g, 15 minutes, 4° C.) and the supernatant was incubated at 4° C. for 2 hours with 25 μL streptavidin beads (pre-equilibrated with RIPA buffer) and briefly centrifuged. The supernatant was removed and an aliquot run on a SDS-PAGE gel for western blot analysis of unbound (i.e., unbiotinylated) CFTR-biotintag. Beads were washed five times with RIPA buffer and adsorbed proteins were eluted by boiling for 3 minutes, centrifuged briefly, and loaded on a SDS-PAGE gel. To isolate chemically biotinylated CFTR-biotintag, cells were exposed to sulfo-NHS-SS-biotin as described herein, washed three times with cold quenching buffer (192 mM glycine, 25 mM Tris, pH 8.3), and solubilized on ice in 1 mL RIPA buffer supplemented with protease inhibitors for 20 minutes and used for western blotting as described herein.

Capture of biotinylated protein on streptavidin beads was compared with that obtained by affinity purification of the same CFTR-biotintag-10His construct using Ni²⁺-NTA (Zhu, et al. (1999) J. Biol. Chem. 274:29102-29107) and using immunopurification on antibody-Protein G beads. Briefly, cells were rinsed, scraped into HEPES buffer, and homogenized by hand with sucrose buffer. The homogenate was centrifuged at 16,000×g for 10 minutes, and supernatant proteins were solubilized by addition of TRITON X-100 to a final concentration of 1% and incubated with Ni²⁺-NTA beads for 2 hours with mixing. After washing the beads to remove non-specifically bound proteins, CFTR was eluted into 60 μL and aliquots loaded on SDS-PAGE gels. A similar procedure was followed for immunopurification, except M3A7 antibody replaced Ni²⁺-NTA beads and the antigen-antibody complexes were bound to protein G beads. These were washed with HEPES/TRITON X-100 buffer, suspended SDS-PAGE loading buffer, boiled for 3 minutes, centrifuged, and loaded on SDS-PAGE gels as described herein. Proteins were transferred to PVDF membranes, and western blots were probed with anti-R domain monoclonal antibody 450 at 1:5,000 dilution.

### EXAMPLE 7

### Imaging Biotinylated CFTR

Confocal microscopy was performed by labeling CFTR-biotintag with fluorescent streptavidin. For static images, biotinylated cells were fixed with 2% paraformaldehyde for 20 min at 22° C., incubated with 1% BSA for 30 minutes, and exposed to streptavidin-Alexa 488 in the dark for 15 minutes. Cells were rinsed with PBS to remove unbound streptavidin and viewed using a Zeiss LSM 510 confocal microscope. To study the lateral diffusion, live cells were incubated with BirA at 30° C. for 40-60 min, briefly exposed to streptavidin-Alexa568 at 4° C., washed, and rewarmed to 230C or to 37° C. using a temperature-controlled chamber (FCS2, Bioptechs Inc., Butler, Pa.). Images were collected with an Olympus FV300, IX71 confocal laser scanning microscope using the 543 nm laser line of a 1 mW He—Ne laser for illumination, and a DMS570 dichroic mirror in combination with a FV3-BA 575-563 nm band pass filter. Fluorescence emitted by Alexa568-conjugated streptavidin was collected by a 60X PlanApo oil immersion objective (NA 1.4) through a 100 μm diameter pinhole. Individual cells were viewed with an electronic zoom that gave a resolution between 0.06 and 0.15 μm/pixel in both x and y directions. Time series of 100-125 images were collected with approximately 1 second between consecutive scans. Control experiments were performed using biotinylated cells that had been fixed by a 20 minute exposure to 2% paraformaldehyde.

### EXAMPLE 8

### Image Correlation Spectroscopy

Images from a 32 by 32 pixel sub-region within the image series were used for analysis. A generalized spatio-temporal intensity fluctuation function was defined as:

$$r ( ζ , η , τ ) = \frac{〈 {δ}_{i} ( x + ζ , y + η , t + τ ) {δ}_{i} ( x , y , t ) 〉}{{〈 i 〉}^{2}} \qquad ( 1 )$$

(Wiseman, et al. (2000) J. Microsc. 200:14-25) where ζ and η are the spatial lag coefficients in the x and y directions, and τ is the time delay between images (temporal lag coefficient). For zero temporal lag, spatial autocorrelation functions were calculated for each image in the time series and fitted to a Gaussian function by nonlinear least squares fitting:
r(ζ,η0)=g(0,0,0)e^{-(ζ}^{²}^{+η}^{²} ^{)/}w^{²}+g₅₀₀ (2)
where the fitting parameters are the zero lag spatial autocorrelation function amplitude g(0,0,0), the e ⁻² beam radius in the focal plane ω, and the spatial offset parameter gs_{s∞} at long correlation lengths. For zero spatial lag, a temporal autocorrelation function was calculated for each time series and fit to a two dimensional diffusion model:

$$r ( 0 , 0 , τ ) = g ( 0 , 0 , 0 ) {( 1 + \frac{τ}{{τ}_{d}} )}^{- 1} + {g}_{t ∞} \qquad ( 3 )$$

Diffusion coefficients were calculated using the average value of the best-fit beam radius for each time series and the characteristic diffusion time, rd, which is one of the best-fit parameters of equation (3):

$$D = \frac{{〈 w 〉}^{2}}{4 {τ}_{d}} \qquad ( 4 )$$

The mobile and immobile fractions were calculated using gs_{t∞}. All correlation calculations and fitting of spatial autocorrelation functions were performed in a LINUX environment using programs written in FORTRAN. Temporal autocorrelation functions were fitted by a nonlinear least squares routine using Sigmaplot for Windows.

1. A method for labeling a membrane-localized protein in a cell comprising introducing a biotin target sequence tag into at least one loop domain of a membrane-localized protein and exposing said tagged protein to a biotin ligase in the presence of biotin so that the membrane localized protein is labeled.

2. The method of claim 1, wherein the membrane-localized protein is an ion channel.

3. The method of claim 2, wherein the membrane-localized protein is cystic fibrosis transmembrane conductance regulator.

4. The method of claim 3, wherein the cystic fibrosis transmembrane conductance regulator has a defect in membrane localization.

5. An isolated recombinant cystic fibrosis transmembrane conductance regulator protein comprising a biotin target sequence tag introduced into an extracellular loop of the cystic fibrosis transmembrane conductance regulator protein encoded by a nucleic acid sequence of SEQ ID NO:1.

6. The recombinant protein of claim 5, wherein the extracellular loop is loop four of the cystic fibrosis transmembrane conductance regulator protein encoded by a nucleic acid sequence of SEQ ID NO:1.

7. A method for identifying an agent which corrects protein misfolding of a membrane-localized protein comprising
obtaining a cell which expresses a misfolded membrane-localized protein, wherein said protein is tagged with a biotin target sequence;
contacting the cell with a test agent and a biotin ligase in the presence of biotin so that the biotin target sequence tag of the protein is labeled; and
detecting the presence of labeled protein in cells contacted with the test agent, wherein the presence of labeled protein indicates the agent corrects protein misfolding of a membrane-localized protein.

8. The method of claim 7, further comprising the step of contacting the cell with a permeabilizing agent before the step of detecting the presence of the labeled protein.
